(19) United States
(12) Patent Application Publication
(10) Pub. No.: US 2025/0040794 A1
(43) Pub. Date: Feb. 6, 2025

(54) ENDOSCOPIC TUBULAR MINIMALLY INVASIVE SURGICAL SYSTEM

(71) Applicant: BOARD OF REGENTS, THE UNIVERSITY OF TEXAS SYSTEM, Austin, TX (US)

(72) Inventors: Justin E. BIRD, Houston, TX (US); Claudio TATSUI, Houston, TX (US)

(21) Appl. No.: 18/722,475

(22) PCT Filed: Dec. 20, 2022
(86) PCT No.: PCT/US2022/082037

§ 371 Date: Jun. 20, 2024

Related U.S. Application Data
(60) Provisional application No. 63/292,312, filed on Dec. 21, 2021.

(51) Int. Cl. A61B 1/01; A61B 1/313

(52) U.S. Cl. CPC A61B 1/01; A61B 1/3132

(57) ABSTRACT

A surgical access port, comprising: an elongated working channel with a lower distal end opening positionable near an internal surgical site and an upper proximal opening that receives surgical instruments in use, the working channel defining an internal hollow cavity with a diameter configured to receive more than one surgical instrument at a time and that allows surgical instruments to be inserted at varying angles; and a plurality of side channels extending along an outer perimeter of the working channel, each side channel defined by an inner-facing wall and an outer wall.

### RELATED APPLICATIONS

This application claims priority to and the benefit of U.S. Provisional Patent Application Ser. No. 63/292,312, filed Dec. 21, 2021, the contents of which is incorporated herein by this reference as if fully set forth herein.

### FIELD

According to certain embodiments of this disclosure, there is provided an improved working channel for endoscopy or other minimally invasive surgery. The working channel provides a main tubular channel access port that is sized to receive more than one instrument at a time, with additional side channels or ports for irrigation, suction, and/or imaging.

### BACKGROUND

Endoscopic surgery is typically performed using an endoscope, which is a tubular instrument with a camera and light at the tip. This allows the surgeon to see the surgical site and perform procedures without making major incisions. If necessary, tools may be inserted through the endoscope to take biopsies, remove tissue, or conduct minimally invasive surgery (MIS). Alternatively, tools may be inserted through a separate single working channel positioned within the patient that receives and guides the various tools required for the MIS. Current designs on the market use a single channel as the conduit to receive multiple tools-tools for visualizing structures as well as the surgical tools needed to accomplish the necessary surgical task. This forces the surgeon's visualization of the surgical tool to be fixed, limiting the instrument movement to just in/out, requiring the entire endoscope system to move in antero-posterior and medio-lateral directions when visualization of different angles of the surgical target is required during the operation. With current endoscopic systems, there is just one working channel and just one instrument can be used. In these systems, the channel may have such a small diameter that only one tool at a time can be inserted into the single channel, requiring insertion of a tool, use of the tool, then retraction of the tool from the channel and insertion of a second tool in order to complete the desired steps at the surgical site. This can add time and complexity to the MIS procedure. Improved working channels for guiding surgical instruments to an internal surgical site are thus desirable.

### BRIEF SUMMARY

Accordingly, the present inventors have designed an improved large diameter working channel for endoscopy or other MIS that provides a main tubular access channel or port, with at least one (but typically three) additional side channels or ports. The additional side channels or ports are generally envisioned as being usable as any or all of a (1) suction port, (2) an irrigation port, and/or (3) a camera port. Larger instruments for conducting the surgery itself may be inserted through the internal primary large working channel. This allows the surgeon a clear view of the surgical site, the ability to apply irrigation and suction at the same time, as well as the ability to insert multiple instruments and have freedom of motion in antero-posterior, medio-lateral planes without changes in the angle of the camera or movement of the system. This facilitates surgical maneuvers through the main working channel without losing visualization of the surgical target or causing collisions between the surgical instruments and the camera.

In certain embodiments, there is provided a surgical access port, comprising: an elongated working channel with a lower distal end opening positionable near an internal surgical site and an upper proximal opening that receives surgical instruments in use, the working channel defining an internal hollow cavity with a diameter configured to receive more than one surgical instrument at a time and that allows surgical instruments to be inserted at varying angles; and a plurality of side channels extending along an outer perimeter of the working channel, each side channel defined by an inner-facing wall and an outer wall.

There may be provided at least three side channels, comprising a suction channel, an irrigation channel, and a camera channel. The irrigation channel can also comprise an irrigation port. There may be provided a built-in camera along an end or outer side wall of the channel.

Various dimensions are possible. For example, the internal hollow cavity may have a diameter of about 8-35 mm. The internal hollow cavity may have a diameter of up to about 60 mm. The elongated working channel may have a length of about 35-130 mm.

It is possible for the inner-facing wall of each side channel to terminate before the lower distal end opening.

The surgical access port may be made of a rigid material that does not bend or flex in use.

Embodiments also provide a method for accessing an internal surgical site, comprising: providing a surgical access port as described herein. The surgical access port may have at least three side channels and in use, the steps may include
inserting the lower distal end opening through an incision to a position at or near the internal surgical site;
inserting a plurality of instruments through the elongated working channel, with more than one instrument insertable at a time;
delivering suction through a first side channel;
delivering irrigation through a second side channel; and
delivering an endoscope camera through a third side channel.

The terms “invention,” “the invention,” “this invention” “the present invention,” “disclosure,” “the disclosure,” and “the present disclosure,” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

### BRIEF DESCRIPTION OF THE DRAWINGS

FIG. 1 is a side transparent perspective view of one embodiment of a surgical access port described herein.

FIG. 2A is a top plan view of the surgical access port of FIG. 1.

FIG. 2B is a top plan view of the surgical access port of FIG. 4.

FIG. 3 is a bottom plan view of the surgical access port of FIG. 1.

FIG. 4 is a side plan view of a surgical access port showing an irrigation port in place and instruments positioned through the side channels.

FIG. 5 is a side transparent perspective view of the surgical access port of FIG. 4.

FIG. 6 is a bottom perspective view of the surgical access port of FIG. 1 showing side channels ending before the distal end and with an endoscope positioned in one of the side channels.

FIG. 7 is a top perspective view of a surgical access port having a sealing cap.

FIG. 8 is a side perspective view of an alternate sealing cap.

### DETAILED DESCRIPTION

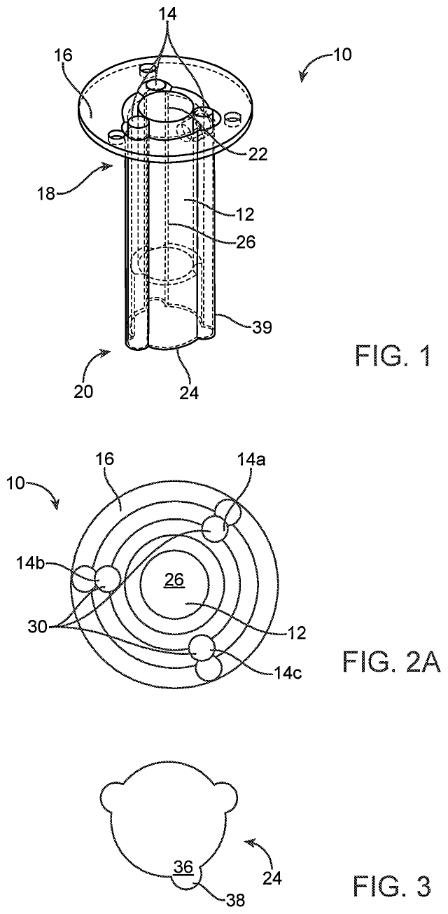

Embodiments of the present disclosure provide an improved working channel for use during endoscopy or other minimally invasive surgeries (MIS). As shown by FIG. 1, there is provided a surgical access port 10 that is defined by an elongated working channel 12, a plurality of side channels 14 (illustrated as 14a, 14b, and 14c), and a support flange 16. The support flange 16 is positioned along an upper portion 18 of the working channel 12. A lower portion 20 of the working channel is configured to be positioned at or near the surgical access site. The working channel 12 has a length sufficient to allow the upper portion 18 to be positioned outside patient's body, with an upper proximal opening 22 that receives one or more surgical instruments, and a lower distal end opening 24 of the lower portion 20 to be positioned at or near the surgical site. In a specific example, the length of the working channel may range from about 35 mm to about 130 mm. It should be understood, however, that any appropriate length depending upon the surgery to be completed is possible and considered within the scope of this disclosure.

An internal hollow cavity 26 of the working channel 12 extends between the upper opening 22 and the lower opening 24. The internal hollow cavity 26 is sized to receive more than one surgical instrument at a time. The diameter of the internal hollow cavity 26 of the working channel 12 can be slightly larger/wider than typical single working channels, allowing more freedom of movement and flexibility for instruments positioned therein, rather than the simple in and out only movement allowed via use of an endoscope.

In a specific example, the internal diameter of the internal hollow cavity 26 may be about 8-35 mm. Other diameters are possible for different types of surgeries. For example, for spinal or pelvic procedures, the diameter of the internal hollow cavity 26 may be about 50-60 mm. Again, it should be understood that any appropriate diameter depending upon the surgery to be completed is possible and considered within the scope of this disclosure. For example, an appropriate internal diameter for a working channel 12 intended for use during a spinal surgery will be much less than an appropriate diameter for a working channel 12 intended for use during a pelvic surgery, which could be up to a few inches if needed. One distinguishing feature of this disclosure is that the diameter of the internal hollow cavity 26 should be such that it can receive more than one surgical instrument at a time. For example, this can allow a surgeon to angle multiple surgical instruments at various planes in order to reach various different angles at the surgical site. For example, a first instrument can move within the working channel 12 in an “X” plane, and a second instrument can move within the working channel 12 in a “Z” plane. Having a large enough internal diameter for this maneuverability allows a single incision to be made for a single surgical access port 10, allowing multiple instruments to be used therethrough. Even if this single incision is larger than the size of a typical incision for an endoscopy, having only a single incision scar can be preferable from a patient standpoint.

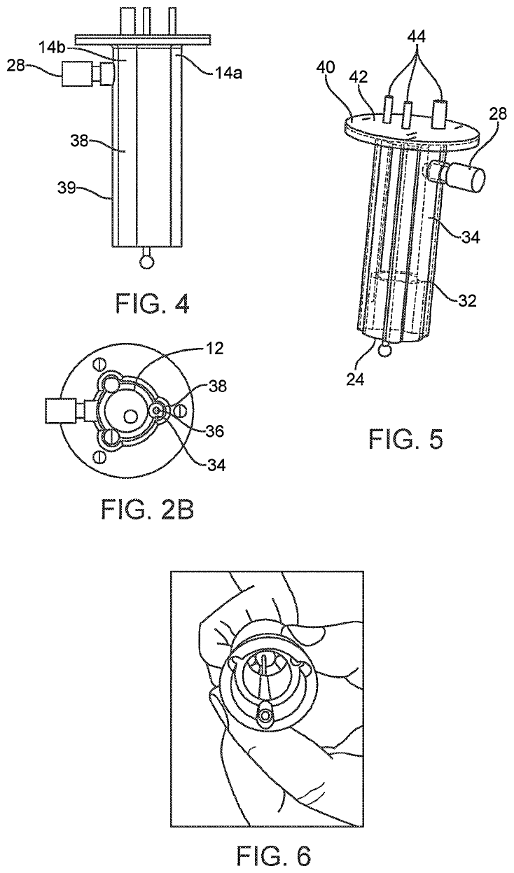

FIGS. 1 and 2 also illustrate a plurality of side channels 14 extending alongside the working channel 12. Although three side channels 14 (14a, 14b, and 14c) are illustrated, it should be understood that more or fewer side channels may be provided. In the embodiment shown, a first side channel 14a may function as a suction channel. A second side channel 14b may function as an irrigation channel. A third side channel 14c may function as a camera channel. The camera channel 14c may receive a traditional elongated endoscope camera. In other embodiments, the camera channel 14c may have a built-in camera.

As shown by FIGS. 4 and 5, the side channel 14 used as an irrigation channel may have a side irrigation port 28. A source of irrigation may be secured to the irrigation port 28 such that fluid can be delivered down the irrigation channel 14b to the surgical site. Delivery of irrigation this way can help distend the surgical site for ease of viewing, can help rinse the camera, or can provide any other necessary need for irrigation. Irrigation can completely flood the surgical port and be drained continuously, creating a sustained constant irrigation flow that creates controlled pressure, decreasing bleeding in vascular channels, removing tissue debris and blood clots, and allowing always clear visualization of the surgical target by the camera. In order to remove the irrigation fluid delivered to the surgical site through the irrigation channel 14b, one or more suction devices may be delivered through the suction channel 14a. The one or more suction devices can be used to suction out irrigation fluid, removed polyps or tissue, or can provide any other necessary need for suction.

The top plan view of FIGS. 2A and 2B illustrates side channel upper openings 30. It is generally envisioned that the diameter of each side channel 14 remain generally constant from its upper opening 30 to its lower opening 32. (However, it should be understood that the diameter may taper or create a funnel-like diameter, such that the diameter of the upper opening 30 is smaller than a larger diameter at the lower opening 32. This may allow for more maneuverability of the instruments positioned within the side channels 14.) Each side channel 14 also has an inner-facing wall 34 and an outer wall 38, which defines an internal cavity 36 of each side channel 14.

Although it is possible for the side channels 14 to extend all the way down to the lower distal end opening 24 of the working channel 12, it is believed that additional maneuverability/flexibility may be achieved if inner-facing walls 34 of the side channels 14 do not extend completely to the lower distal end opening end 24 of the working channel 14. This feature is illustrated by the transparent views of FIGS. 1 and 5 and the top side perspective view of FIG. 6. In these figures, it can be seen that each side channel 14 has an inner-facing wall 34 that terminates at a lower opening 32 before the lower distal end opening 24. This results in the bottom plan view of FIG. 3, showing the outer walls 38 of the side channels 14 at the lower distal end opening 24 and the semi-circle shaped/partial internal cavity 36 that results at the lower distal end 24 of the working channel 12 from the lack of inner-facing walls 34 of side channels 14 extending all the way down. As shown, the outer walls 38 of the side channels 14 also define an outer perimeter 39 of the working channel 12, such that outer perimeter 39 and outer walls 38 are shared walls. The side plan view of FIG. 4 shows that the outer walls 38 of the side channels 14 do extend all the way to the lower distal end 24 of the working channel 12.

Although it is generally envisioned that each of the side channels 14 will have similar diameters, it is also possible for the side channels to have varying diameters with respect to one another.

As described above, the camera channel 14c may receive a separate endoscopic camera. Alternatively, a camera may be built into the end of one of the side channels such that it functions as a camera channel 14c. Alternatively, a camera may be built into the end of the main working channel 12 itself, rather than requiring insertion of an endoscope through one of the side channels 14. The camera can connect to a cable that extends through a side of the working channel 12, so that nothing obstructs the working/upper surface of the tube. The camera may be removable or disposable.

As described above, the length of the elongated working channel 12 may be varied depending upon the surgery for which it is intended to be used. Additionally, the diameter of the elongated working channel 12 may similarly be varied. In one example, the entire surgical access port 10 may be 3D printed so that it is customizable for various depths (lengths) or surgical locations (diameters).

It is generally envisioned that the surgical access port 10 is a rigid structure that does not bend or flex in use. Possible materials include but are not limited to high density polyethylene (HDPE), aluminum, titanium, PEEK, carbon fiber, stainless steel, or any other appropriate biocompatible materials, or any combination thereof.

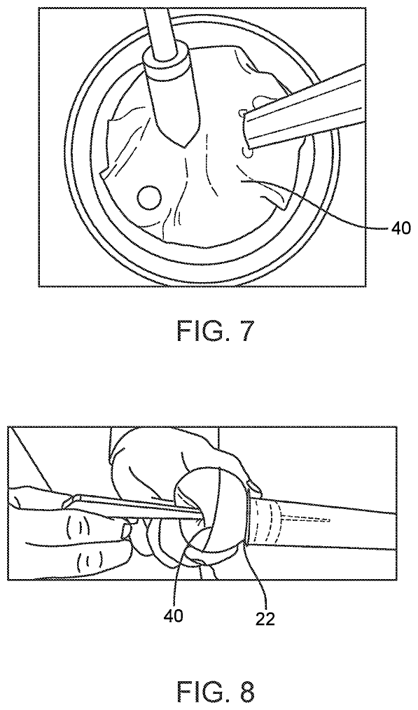

It is also possible to provide a sealing cap 40 positionable over the top of the working channel 12 in order to close the working channel 12 and maintain a positive pressure therein. The sealing cap may be a flat silicone or rubber cover 42 as illustrated by FIG. 5. In this embodiment, the silicone or rubber cover 42 may have a plurality of openings 44 that receive various instruments therethrough. FIG. 7 illustrates an alternate option of a sealing cap 40, which is a dome shaped cap with a valve system that can allow introduction of multiple instruments. In some embodiments, it may be made of silicon/rubber material.

FIG. 8 illustrates a further alternate option of a sealing cap 40. In this example, the sealing cap is a syringe bulb that is positioned over the upper proximal opening 22 to function as a seal.

It is also possible to provide a handle or other type of connector that extends from the side of the working channel 12 so that the channel 12 can be connected to a surgical table or other stationary surface for stabilization.

It is further possible to provide a seal around the interface between the working channel 14 and the patient's skin to create a seal for suction and/or to prevent fluid leakage at the interface.

The subject matter of certain embodiments of this disclosure is described with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

It should be understood that different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

What is claimed is:

1. A surgical access port, comprising:
(a) a support flange configured to be positioned exterior to a patient;
(b) an elongated body configured to extend through an incision in the patient towards a surgical site;
(c) a multi-instrument hollow working channel extending from the support flange through the elongated boy, the working channel comprising an open proximal end in the support flange and an open distal end at a distal end of the elongated body;
(d) a plurality of side channels extending through the elongated body at least partially along the working channel, wherein the plurality of side channels are at least partially separated from the working channel.

2. The surgical access port of claim 1, wherein the side channels merge with the working channel proximate to the open distal end.

3. The surgical access port of claim 2, wherein the working channel is wider distal to where the side channels merge with the working channel.

4. The surgical access port of claim 2, wherein at least one of the plurality of side channels comprises an open proximal end in the support flange.

5. The surgical access port of claim 4, wherein at least one of the plurality of side channels comprises a proximal port extending through a side wall of the elongated body.

6. The surgical access port of claim 5, wherein the proximal port comprises an irrigation port configured to connect to a water source.

7. The surgical access port of claim 1, wherein the working channel has a diameter that is at least twice a diameter of each of the side channels.

8. The surgical access port of claim 7, wherein a diameter of a first side channel of the plurality of side channels is different from a diameter of a second side channel of the plurality of side channels.

9. The surgical access port of claim 7, wherein a diameter of a first side channel of the plurality of side channels is the same as a diameter of a second side channel of the plurality of side channels.

10. The surgical access port of claim 1, wherein the working channel is configured to allow visualization of the surgical site from the open proximal end.

11. The surgical access port of claim 1, further comprising a sealing cap configured to seal at least the open proximal end of the working channel from an ambient environment external to the patient.

12. The surgical access port of claim 1, further comprising a seal configured to form a seal between at least one of the flange and the body with the patient.

13. A surgical access port (10), comprising:
an elongated working channel (12) with a lower distal end opening (24) positionable near an internal surgical site and an upper proximal opening (22) that receives surgical instruments in use, the working channel defining an internal hollow cavity (26) with a diameter configured to receive more than one surgical instrument at a time and that allows surgical instruments to be inserted at varying angles; and
a plurality of side channels (14) extending along an outer perimeter (39) of the working channel (12), each side channel defined by an inner-facing wall (34) and an outer wall (30).

14. The surgical access port of claim 13, comprising at least three side channels (14), comprising a suction channel, an irrigation channel, and a camera channel.

15. The surgical access port of claim 13, wherein the irrigation channel further comprises an irrigation port (28).

16. The surgical access port of claim 13, further comprising a built-in camera.

17. The surgical access port of claim 13, wherein the internal hollow cavity comprises a diameter of about 8-35 mm.

18. The surgical access port of claim 13, wherein the internal hollow cavity comprises a diameter of up to about 60 mm.

19. The surgical access port of claim 13, wherein the elongated working channel (12) has a length of about 35-130 mm.

20. The surgical access port of claim 13, wherein the inner-facing wall (34) of each side channel terminates before the lower distal end opening (24).

21. The surgical access port of claim 13, comprising a rigid material that does not bend or flex in use.

22. A method for accessing an internal surgical site, comprising:
(a) providing the surgical access port of claim 13, wherein the surgical access port comprises at least three side channels (14);
(b) inserting the lower distal end opening (24) through an incision to a position at or near the internal surgical site;
(c) inserting a plurality of instruments through the elongated working channel (12), with more than one instrument insertable at a time;
(d) delivering suction through a first side channel;
(e) delivering irrigation through a second side channel;
(f) delivering an endoscope camera through a third side channel.
